(19) United States
(12) Patent Application Publication
(10) Pub. No.: US 2005/0131275 A1
(43) Pub. Date: Jun. 16, 2005

(54) Catheter for intrauterine insemination

(76) Inventor: Juan Jose Mena Gerico, Zaragoza (ES)

Correspondence Address: Klauber & Jackson;David A. Jackson, Esq., 4th Floor, 411 Hackensack Ave., Hackensack, NJ, 07601, US

(21) Appl. No.: 11/000,529

(22) Filed: Nov. 30, 2004

Related U.S. Application Data
(63) Continuation of application No. PCT/ES03/00131, filed on Mar. 20, 2003.

(30) Foreign Application Priority Data
Jun. 3, 2002 (ES) P200201269

(51) Int. Cl. A61B 17/43

(52) U.S. Cl. 600/35; 604/275

(57) ABSTRACT

Catheter for intrauterine insemination, which is useful in artificial insemination in different animal species and, thus, it is preferably useful for intrauterine insemination of pigs. The catheter (1) for intrauterine insemination is defined by a unitary element comprised of an elongated tube-shaped body (2) provided in one of its ends, the end to be introduced in the reproductive system of the animal, with a rounded surface body (3) provided with a hole (4) as an extension of the axial hole (5) of the tube-shaped body (2).

### RELATED APPLICATIONS

The present application is a Continuation of co-pending PCT Application No. PCT/ES03/00131, filed Mar. 20, 2003 which in turn, claims priority from Spanish Application Serial No. 200201269, filed on Jun. 3, 2002. Applicants claim the benefits of 35 U.S.C. §120 as to the PCT application and priority under 35 U.S.C. §119 as to said Spanish application, and the entire disclosures of both applications are incorporated herein by reference in their entireties.

### OBJECT OF THE INVENTION

As expressed in the title of the present specification, the following invention, refers to a catheter for intrauterine insemination. It is preferably useful for intrauterine insemination of pigs, in such a way that the catheter is defined by a unitary element comprised of an elongated tube-shaped body provided in one of its ends, specifically the end to be introduced into the reproductive system of the animal, with a rounded surface body provided with a hole as an elongation of the axial hole of the tube-shaped body.

The catheter that is described has as the main novelty the fact that it is a catheter for intrauterine insemination defined by a unitary element by means of which access to the body and/or uterine horns is achieved, for the purpose of depositing the insemination dose in said place.

Besides, by means of the described catheter, the process involved in intrauterine insemination of pigs is simplified, since, in a single act one manages to reach the place to deposit the insemination dose, which in turn involves a saving in the time used for this activity.

### FIELD OF USE

The present catheter for intrauterine insemination is useful for artificial insemination of very different animal species, and, thus, it is useful for insemination of sheep, cattle, horses, etc. and is especially useful for pigs.

### BACKGROUND OF THE INVENTION

Basing ourselves on the conventional artificial insemination technique used for pigs, we can state that same consists of introducing in the first part of the uterine cervix a number of spermatozoa equal to or higher than 3 billion in an insemination volume of less than 50 ml.

Use of a lower number of spermatozoa causes a reduction of fertility unless, the deposit is made in an innermost place inside the genital tract.

Up to now, in order to make the deposit in the innermost place of the genital tract, it has only been possible to do so by means of anesthetizing the animal and using complicated surgical techniques. As a result thereof, intrauterine insemination of sows is difficult due to the presence of a relative long cervix with cartilaginous folds.

On the other hand, the systems for intrauterine insemination of pigs that exist on the market are characterized in that they are comprised of two parts: a guiding catheter and a flexible or semi-rigid probe.

The guiding catheter is commonly defined by a tube-shaped body provided in one of its ends, with a generally conical or cylindrical-shaped body provided with an axial hole as an extension of the axial hole of the tube-shaped body which is inserted in the genital tract of the animal. The purpose of the guiding catheter is to find the external cervical hole, inserting itself through it and attaching to the caudal part of the cervix.

On the other hand, the different types of flexible or semi-rigid probes are inserted through the internal hole of the guiding catheter in such a way that they project into the lumen of the cervix in its middle part, so that from there, and by means of the propelling force that is exerted from the outside, they are capable of passing through the rest of said structure up to the uterine body and/or horns.

### DESCRIPTION OF THE INVENTION

The present specification describes a catheter for intrauterine insemination, preferably useful for intrauterine insemination of pigs, in such a way that the catheter for intrauterine insemination is defined by a unitary element comprised of an elongated tube-shaped body provided in one of its ends, specifically, the end to be inserted into the reproductive system of the animal, with a rounded surface body provided with a hole as an extension of the axial hole of the tube-shaped body.

The catheter for intrauterine insemination has suitable rigidity in order to have access, through the end provided with a rounded surface body, to the uterine body and/or horns, for the purpose of depositing the insemination dose in said place.

In this way, we obtain a catheter for intrauterine insemination comprised of a single part, which entails economic advantages, since, the materials that it is made of, are logically cheaper than those of a conventional guiding catheter and corresponding probe. Besides, the catheter is also simpler to manufacture and this in turn affects the production cost thereof, and, consequently, the final price of the catheter.

Likewise, since the catheter for intrauterine insemination is comprised of a single part, the process involved in intrauterine insemination of pigs is simplified, since, in a single act the place where the insemination dose is to be deposited is reached. In turn, this involves a saving of time used for this activity.

In order to complete the description that is going to be made hereinafter, and to provide a better understanding of the characteristics of the invention, a set of drawings, in whose figures the most characteristics details of the invention are represented in an illustrative and non-restrictive manner, is attached to the present specification.

### BRIEF DESCRIPTION OF THE FIGURES

FIG. 1 shows a raised view of a catheter for intrauterine insemination, where it is possible to see how the catheter is defined by a tube-shaped body, one of whose ends ends in a generally spherical-shaped body.

FIG. 2 shows a detailed view of the generally spherical-shaped body which the tube-shaped body comprising the catheter for intrauterine insemination, object of the invention, ends in.

FIG. 3 shows a detailed view of another embodiment wherein the rounded surface body which the tube-shaped body comprising the catheter for intrauterine insemination ends in, has an oval shape.

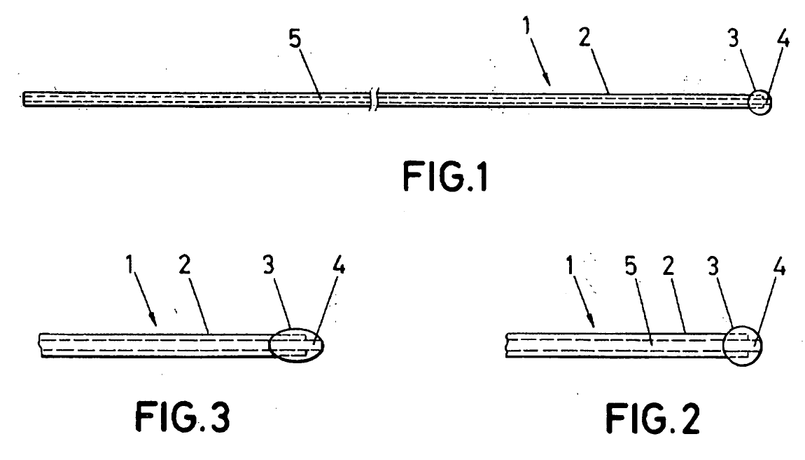

### DESCRIPTION OF A PREFERRED EMBODIMENT

In view of the commented figures and in accordance with the numbering used, we can see how the catheter (1) for intrauterine insemination is defined by a unitary element comprised of an elongated tube-shaped body (2) provided in one of its ends, specifically the end to be introduced in the reproductive system of the animal, with a rounded shape body (3) provided with a hole (4) as an extension of the axial hole (5) of the tube-shaped body (2).

Hence, the rounded surface body (3) may be defined by different shapes, and, in this way, it may be defined by a generally spherical-shaped, oval-shaped body or one of any other shape that has a rounded surface to facilitate insertion thereof into the reproductive system of the animal.

On the other hand, the catheter (1) for intrauterine insemination has suitable rigidity in order to have access, through the end provided with a rounded surface body (3), to the uterine body and/or horns, for the purpose of depositing the insemination dose in said place.

Hence, the catheter (1) for intrauterine insemination comprised of a single part, which entails economic advantages, since, the materials that it is made of, are logically cheaper, and the manufacturing thereof is also simpler. This affects the production cost of the catheter, and, consequently, the final price thereof.

Likewise, the catheter (1) for intrauterine insemination has the added advantage that since it is comprised of a single part, the process for intrauterine insemination of pigs is simplified, since, in a single act, one manages to reach the place to deposit the insemination dose, which in turn involves a saving in the time used for this activity.

The catheter (1) for intrauterine insemination is useful to carry out intrauterine insemination in different animal species, and, thus, it is especially applicable to pigs. It is also applicable to sheep, horses, cattle and the like.

1. Catheter for intrauterine insemination, which is useful for artificial insemination of different animal species and hence, it is preferably useful for intrauterine insemination of pigs, wherein the catheter for intrauterine insemination is defined by a unitary element comprised of an elongated tube-shaped body provided in one of its ends, the end to be introduced in the reproductive system of the animal, with a rounded surface body provided with a hole as an extension of the axial hole of the tube-shaped body.

2. Catheter for intrauterine insemination, according to claim 1, wherein the catheter for intrauterine insemination has suitable rigidity in order to have access, through the end provided with a rounded surface body, to the uterine body and/or horns, for the purpose of depositing the insemination dose in said place.
